(19) United States
(12) Patent Application Publication
(10) Pub. No.: US 2010/0318130 A1
(43) Pub. Date: Dec. 16, 2010

(54) FLEXIBLE ROD ASSEMBLY FOR SPINAL FIXATION

(76) Inventors: Brian D. Parlato, Hillsborough, NJ (US); Jeffrey Michael Spivak, Scarsdale, NY (US); Brian Umbach, North Caldwell, NY (US)

Correspondence Address: ARTHUR L. LESSLER, 540 OLD BRIDGE TURNPIKE, SOUTH RIVER, NJ, 08882, US

(21) Appl. No.: 12/679,491

(22) PCT Filed: Dec. 2, 2008
(86) PCT No.: PCT/US08/85223

§ 371 Date: Aug. 27, 2010

Related U.S. Application Data
(60) Provisional application No. 61/014,027, filed on Dec. 15, 2007.

(51) Int. Cl. A61B 17/70

(52) U.S. Cl. 606/254

(57) ABSTRACT

A flexible spinal fixation rod assembly that provides relatively stiff bending compliance provided by a core rod or tube that is connected to surrounding spring and associated couplings in such a way that when the core rod is bent, the spring extends and the associated couplings move away from each other, with the core rod generating a force tending to return the core rod to its unbent configuration thereof and the spring generating a force tending to return the couplings to there unextended configuration thereof. Couplings connect each end of the rod or tube to corresponding pedicle screws for attachment to corresponding vertebrae. As the spine bends, this arrangement allows the spine to bend against limited resistance, exhibiting the necessary axial and compressive strength, and applies force to return the spine to its original configuration.

### CROSS-REFERENCE TO RELATED APPLICATION

This application claims the benefit of U.S. Provisional Application Ser. No. 61/014,027 filed Dec. 15, 2007 and entitled Flexible Shaft For Spinal Fixation.

### BACKGROUND OF THE INVENTION

The present invention relates to a flexible rod assembly for stabilizing the spine while allowing the spine to bend.

For cases of congenital spinal defects (such as scoliosis), degenerative disc disease and spinal trauma, surgical intervention is typically required to stabilize and support the spine and eliminate pain. Such stabilization has traditionally been provided by rigid metal rods positioned along the posterior side of the spine, fabricated from stainless steel or titanium to support, strengthen, and/or straighten the spine. These stabilizing rods are firmly attached to pedicle screws which are fastened to adjacent vertebrae.

Spinal support is required to prevent undue stress on the discs on either side of the vertebrae that are connected to the rod.

One of the main drawbacks to using such rigid metal rods is that they do not allow virtually any bending of the spine and they do not allow axial stretch along the rod axis. The bending and axial stiffness of the metal rods greatly restrict the amount of bending by a patient, since the rods are offset approximately ¾ inch to 1 inch from the actual spine axis (bending axis), requiring extension so as to permit bending. Spinal bending and stretching is required to prevent undue stress on the discs on either side of the vertebrae that are connected to the rod.

Recently, there have been various attempts to use more compliant materials, such as polymers or combinations of polymers in order to allow some flexing or bending of the spine while still providing stabilization. In other embodiments, combinations of metal and polymer have been employed. However, all such prior art arrangements exhibit significant limitations in being able to provide stabilization of the spine while approximating the everyday natural flexion of the spine.

Accordingly, an object of the present invention is to provide a spine stabilizing rod that exhibits a high degree of stability, which has axial compressive strength that allows for moderate bending stiffness and very low axial extension stiffness.

Another object of the invention is to provide a flexible spine stabilizing rod having dimensions similar to those of currently used spine stabilization rods, and requiring general installation procedures similar to those currently used by surgeons for other spine stabilization rods, so as to allow the patient to bend and flex in a near-natural manner, thus allowing a damaged disc or discs to move in a natural motion while still providing spinal support.

### SUMMARY OF THE INVENTION

According to the invention a flexible, bendably resilient spinal fixation rod assembly is provided which has an elongated inner member surrounded by an elongated outer member coaxial with the inner member. One of the members is relatively stiff and the other member is relatively compliant. A first end of the inner member is coupled to an adjacent first end of the outer member so that there is essentially no relative movement between the first ends of the members. A first coupling is operatively associated with the first ends of the members and can be connected to a pedicle screw. A second coupling is operatively associated with the second ends of the members and can be connected to a second pedicle screw so as to allow movement relative to the first pedicle screw. The pedicle screws are adapted to be connected to corresponding vertebrae to stabilize the spine of a patient.

This bendably resilient flexible spinal fixation rod is intended for dynamic stabilization of the spine. According to a preferred embodiment of the invention a flexible spinal fixation rod and spring combination that provides relatively stiff bending compliance has couplings at both ends, one of which couplings (the floating coupling) allows the spring to extend independently of the rod. Each coupling is adapted to be attached to a pedicle screw, with the screws being connected to corresponding vertebrae so as to span two or more vertebrae and allow limited extension axially when the spine is bent. This is important because as the spine bends, it bends around the central axis of the spine, and the spine rod is attached several inches from that axis, so that the arc length of the rod and spring combination must increase as the spine's radius of bending decreases. Due to the spring-like characteristics of the flexible spine fixation rod assembly, it returns to its original configuration so as to help maintain the shape and axial position of the corresponding portion of the spine and provides axial compressive strength to the spine.

### IN THE DRAWING

FIG. 1a is an isometric view of a flexible spinal fixation rod assembly according to a first embodiment of the invention.

FIG. 1b is a front elevation view of the rod assembly shown in FIG. 1a.

FIG. 1c is a front cross-sectional view of the rod assembly shown in FIG. 1a.

FIG. 2a is an isometric view of a flexible spinal fixation rod assembly according to a second embodiment of the invention.

FIG. 2b is a front elevation view of the rod assembly shown in FIG. 2a.

FIG. 2c is a front cross-sectional view of the rod assembly shown in FIG. 2a.

FIG. 3a is an isometric view of a flexible spinal fixation rod assembly according to a third embodiment of the invention.

FIG. 3b is a front elevation view of the rod assembly shown in FIG. 3a.

FIG. 3c is a front cross-sectional view of the rod assembly shown in FIG. 3a.

FIG. 4a is an isometric view of a flexible spinal fixation rod assembly according to a fourth embodiment of the invention.

FIG. 4b is a front elevation view of the rod assembly shown in FIG. 4a.

FIG. 4c is a front cross-sectional view of the rod assembly shown in FIG. 4a.

FIG. 5a is an isometric view of a flexible spinal fixation rod assembly according to a fifth embodiment of the invention.

FIG. 5b is a front elevation view of the rod assembly shown in FIG. 5a.

FIG. 5c is a front cross-sectional view of the rod assembly shown in FIG. 5a.

FIG. 6a is an isometric view of a flexible spinal fixation rod assembly according to a sixth embodiment of the invention.

FIG. 6b is a front elevation view of the rod assembly shown in FIG. 6a.

FIG. 6c is a front cross-sectional view of the rod assembly shown in FIG. 6a.

FIG. 7a is an isometric view of a flexible spinal fixation rod assembly according to a seventh embodiment of the invention.

FIG. 7b is a front elevation view of the rod assembly shown in FIG. 7a.

FIG. 7c is a front cross-sectional view of the rod assembly shown in FIG. 7a.

FIG. 8a is an isometric view of a flexible spinal fixation rod assembly according to a eighth embodiment of the invention.

FIG. 8b is a front elevation view of the rod assembly shown in FIG. 8a.

FIG. 8c is a front cross-sectional view of the rod assembly shown in FIG. 8a.

FIG. 9a is a left side elevation view of the flexible spinal fixation rod assembly shown in FIGS. 1a through 1c in intended or straight configuration attached to two vertebrae spanning one spinal disc.

FIG. 9b is a left side elevation view of the flexible spinal fixation rod assembly shown in FIGS. 1a through 1c in a bent configuration attached to two vertebrae spanning one spinal disc.

FIG. 9c is a left side elevation view of a typical standard flexible spinal fixation rod without the ability to expand axially in a bent configuration attached to two vertebrae spanning one spinal disc.

### DETAILED DESCRIPTION

### First Embodiment

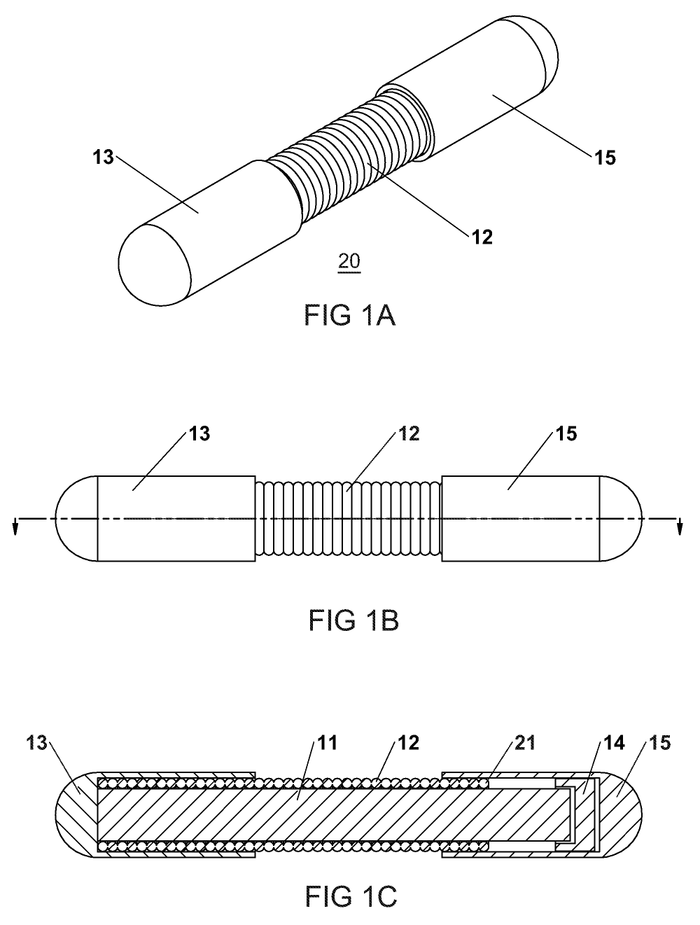

A first embodiment is shown in FIGS. 1a through 1c, in which a flexible spinal fixation bendably resilient rod assembly 20 is adapted to span a single spinal disc and to be fastened at each end to an adjacent vertebra via pedicle screws.

The various rod assembly embodiments described herein are bendably resilient. That is, they resiliently resist bending from their original configurations and return to those original configurations when the bending stress applied to them is relaxed.

The assembly 20 consists of a single length (unitary) flexible inner core rod 11 slidably and coaxially disposed within an outer cylindrical spring 12. A fixed end coupling 13 is positioned over the core 11 and overlaps the adjacent end of spring 12 in such a way as to effectively secure the fixed end coupling 13, adjacent end of core 11, and adjacent end of spring 12 together.

An optional end stop 14 is affixed to the opposite end of core rod 11 to provide a limit to axial travel or sliding of the adjacent end 21 of the spring 12 on the core rod 11. A sliding or floating end coupling 15 is located at this opposite end, positioned over the core 11 and overlapping the adjacent end of spring 12. The floating end coupling 15 is affixed to only the spring 12, thus allowing the spring 12 to extend relative to the core rod 11, that is by sliding along the rod 11; which sliding movement is limited only by engagement of the end 21 of the spring 12 with the end stop 14.

The core rod 11 may be made of any flexible material which is biologically inert, such as titanium, stainless steel or a suitable plastic, and may be solid, hollow, or of wire wound and/or multilayered construction.

### Second Embodiment

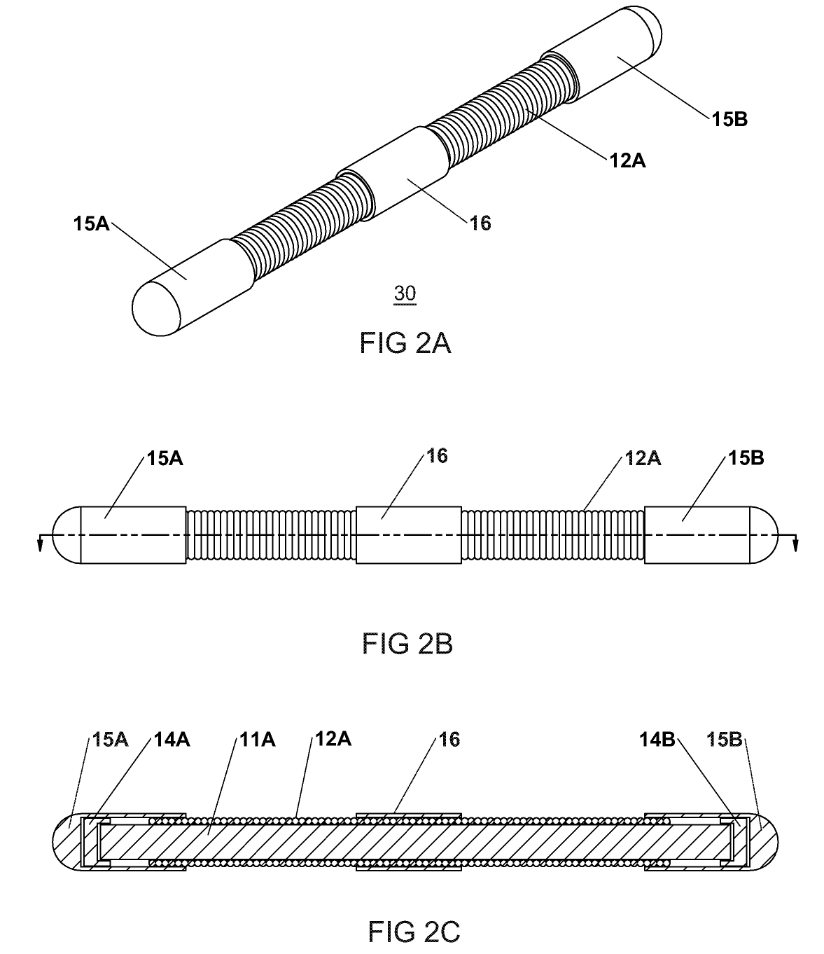

A second embodiment is shown in FIGS. 2a through 2c which shows a rod assembly 30 designed to span three vertebrae (two disc spaces). Its construction and operation is similar to the first embodiment illustrated in FIGS. 1a through 1c, having an inner core rod 11A as described above, but with sliding or floating end couplings 15A and 15B at each end. At the center of the assembly 30 is a fixed coupling 16 which is secured to the adjacent central portions of both the core 11A and spring 12A. The central coupling 16 also provides a clamping location for a middle pedicle screw.

### Third Embodiment

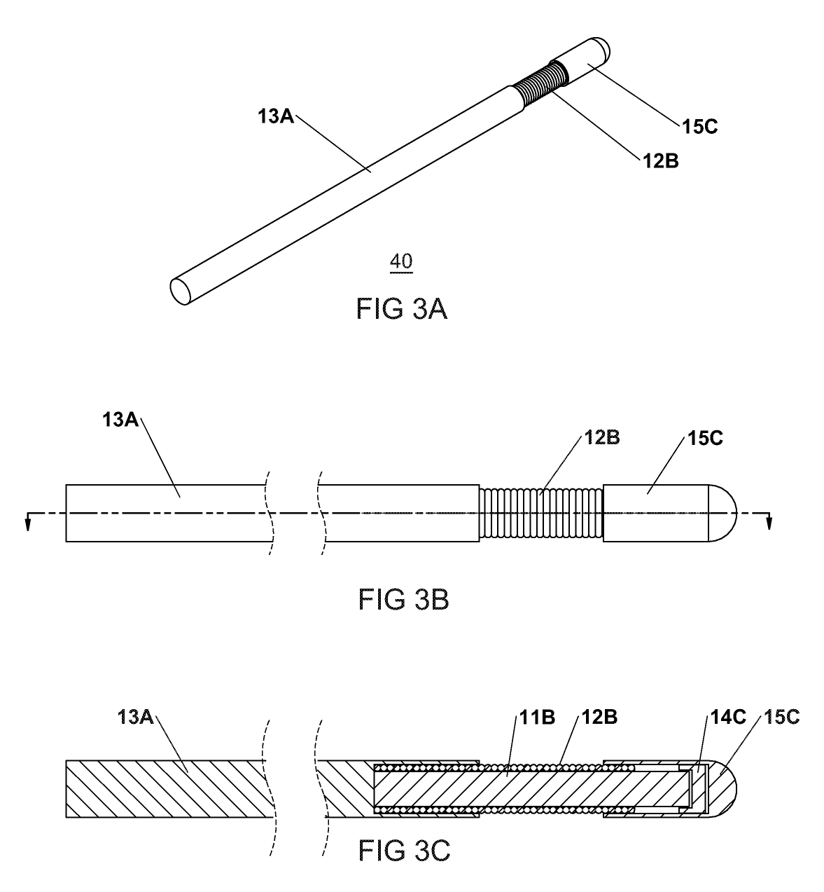

A third embodiment is shown in FIGS. 3a through 3c. The rod assembly 40 is designed to span a single spinal disc space (two vertebrae) and has a construction similar to that of the assembly 20. The assembly 40 includes a fixed end coupling 13A, which is substantially longer than the opposite floating end coupling 15C, for rigid fixation of adjacent spinal discs and vertebrae. The longer fixed end coupling 13A is (except for length) similar to the fixed end coupling 13 shown in FIGS. 1a through 1c, and also serves a purpose similar to that of the fixed center coupling 16 shown in FIGS. 2a through 2c. This third embodiment is suitable for use in patients who have vertebrae which were fused in previous surgery.

### Fourth Embodiment

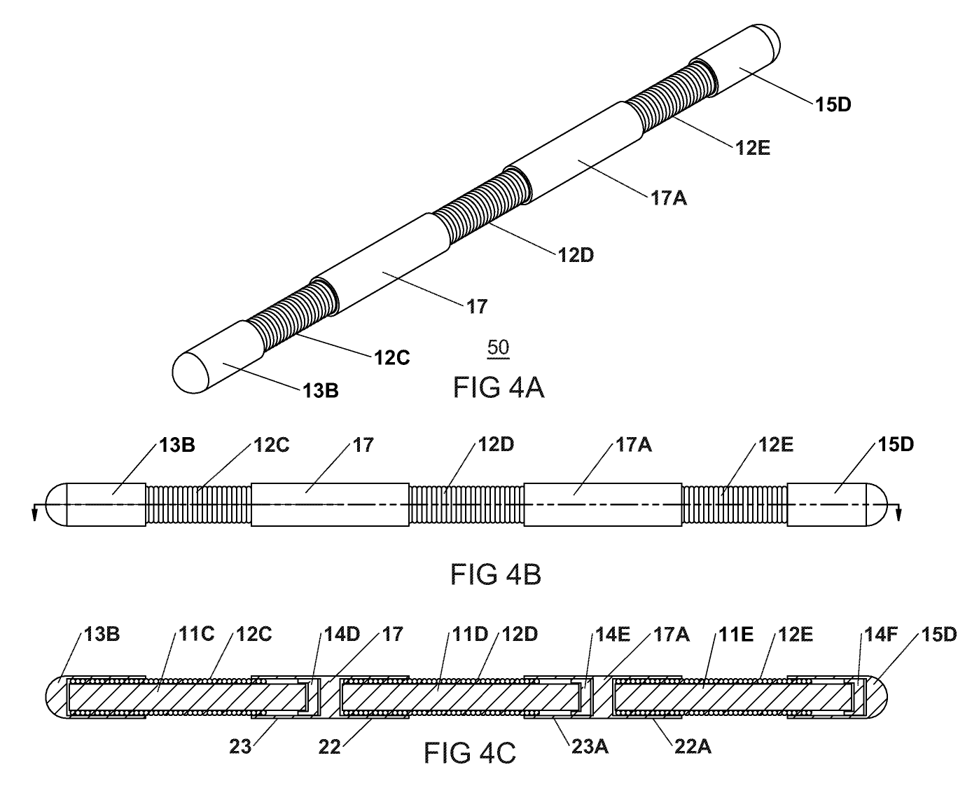

A fourth embodiment is shown in FIGS. 4a through 4c. The flexible fixation rod assembly 50 is designed to span four or more vertebrae (three or more spinal disc spaces) and is similar in construction to the assembly 20, but consists of repeating elements. The construction and operation of the assembly 50 is similar to that of assemblies 20 and 30, with each of the center couplings 17 and 17A now having fixed sections 22 and 22A respectively on one end and sliding sections 23 and 23A on the other end, to allow each of the core rod sections 11C, 11D and 11E to slide or extend individually; the extension of each section being limited by corresponding end stops 14D, 14E and 14F. This embodiment can be extrapolated to any number of vertebrae by adding or subtracting as many repeating elements as are needed. Alternatively, the center couplings 17 and 17A may be lengthened so as to be substantially longer than the end couplings 13B and 15D to rigidly span two or more fused vertebrae between bendably supported sections.

### Fifth Embodiment

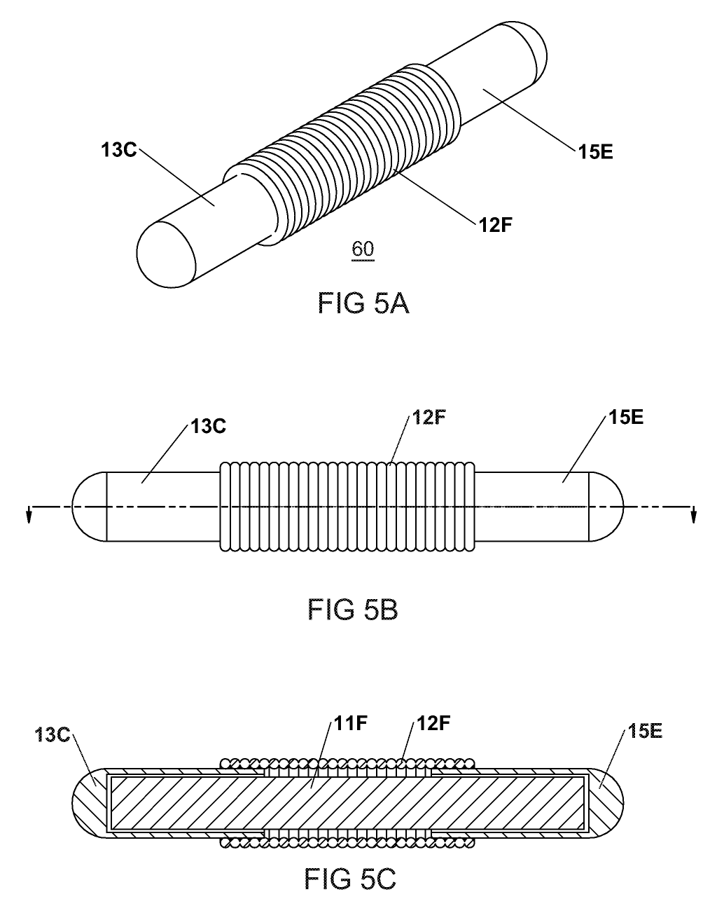

A fifth embodiment is shown in FIGS. 5a through 5c, in which a flexible spinal fixation rod assembly 60 is adapted to span a single spinal disc and to be fastened at each end to an adjacent vertebra via pedicle screws. The rod assembly 60 has a construction similar to that of the assembly 20 except the spring 12F is disposed over or next to the couplings 13C and 15E

The core rod 11F may be made of any resilient and flexible material which is biologically inert, such as titanium, stainless steel or a suitable plastic, and may be solid, hollow, or of wire wound and/or multilayered construction.

### Sixth Embodiment

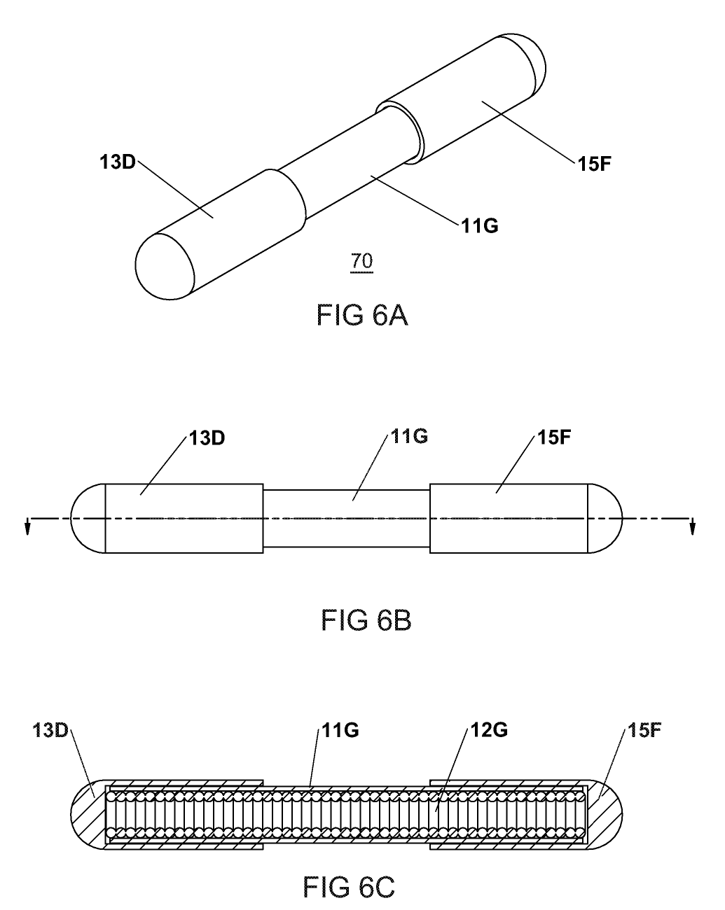

A sixth embodiment is shown in FIGS. 6a through 6c, in which a flexible spinal fixation rod assembly 70 is adapted to span a single spinal disc and to be fastened at each end to an adjacent vertebra via pedicle screws. The rod assembly 70 has a construction similar to that of the assembly 20 except that the spring 12G is disposed inside of tubular resilient core rod 11G and the couplings 13D and 15F are positioned directly over core rod 11G.

The core rod 11G may be made of any flexible material which is biologically inert, such as titanium, stainless steel or a suitable plastic, and may be solid, hollow, or of wire wound and/or multilayered construction.

### Seventh Embodiment

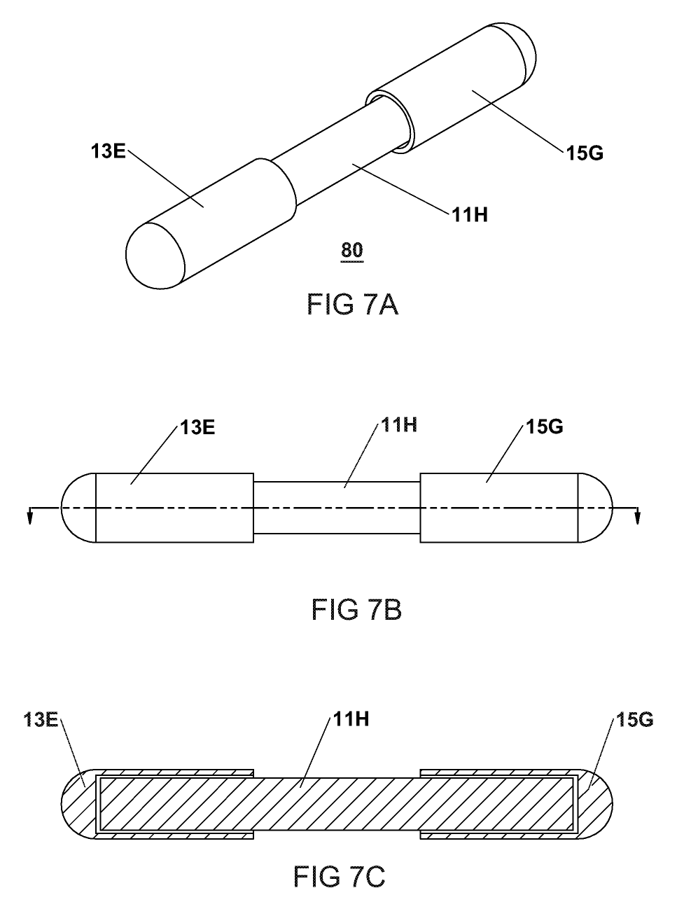

A seventh embodiment is shown in FIGS. 7a through 7c, in which a flexible spinal fixation rod assembly 80 is adapted to span a single spinal disc and to be fastened at each end to an adjacent vertebra via pedicle screws. The rod assembly 80 has a construction similar to that of the assembly 20 except there is no spring over core rod 11F and the couplings 13D and 15F are positioned directly over core rod 11F and function in a manner similar to assembly 20, so coupling 13D will be fixed and coupling 15F will be floating or allowed to move relative to coupling 13D.

The core rod 11F may be made of any flexible, resilient and axially extensible construction, and of a material which is biologically inert, such as titanium, stainless steel or a suitable plastic, and may be solid, hollow, or of wire wound and/or multilayered construction.

### Eighth Embodiment

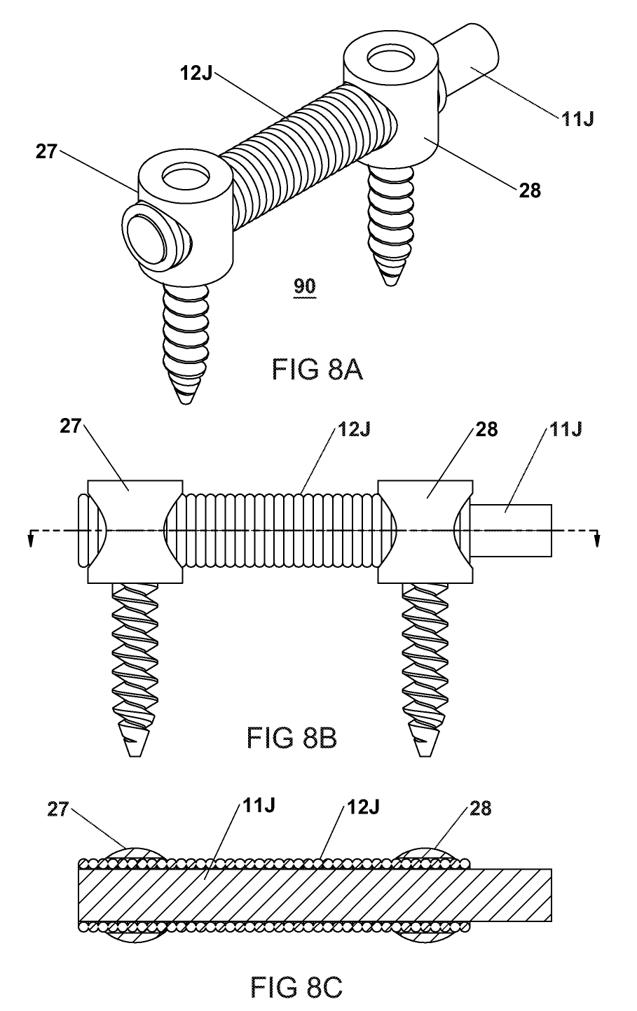

An eighth embodiment is shown in FIGS. 8a through 8c, in which a flexible, resilient and axially extensible spinal fixation rod assembly 90 is adapted to span a single spinal disc and to be fastened at each end to an adjacent vertebra via pedicle screws. The rod assembly 90 has a construction similar to that of the assembly 20 except the pedicle screws may be attached directly to the spring 12J which is fixed at one end to core rod 11J and free to move at the other end.

The core rod 11F may be made of any flexible material which is biologically inert, such as titanium, stainless steel or a suitable plastic, and may be solid, hollow, or of wire wound and/or multilayered construction.

### Installation of the Assembly

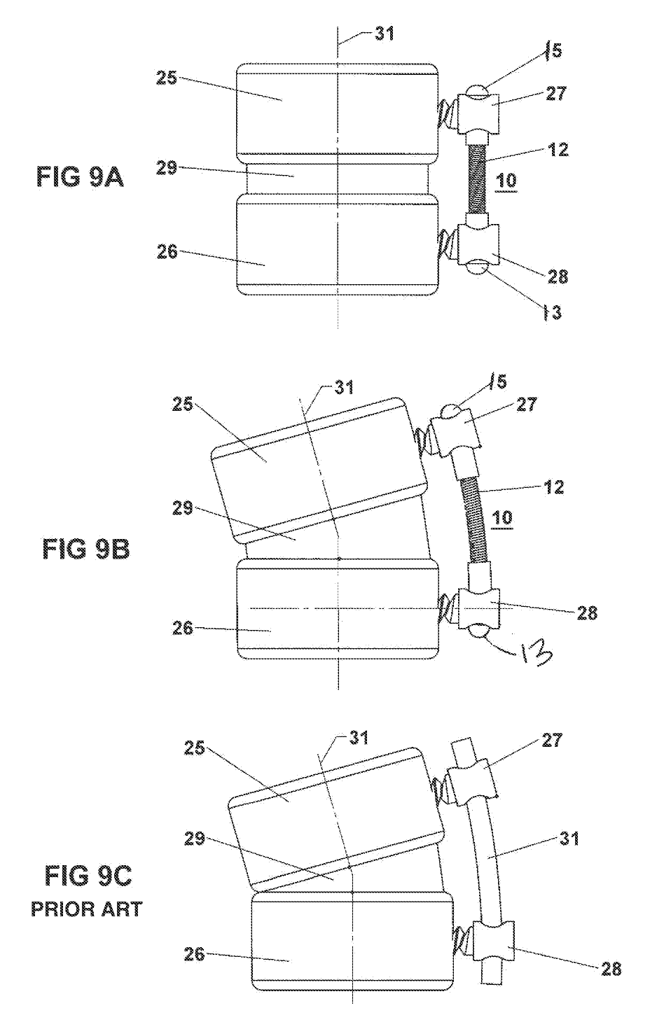

The manner in which the flexible rod assembly is installed adjacent the spine of a patient is illustrated in FIGS. 9a and 9b which show the assembly 10 stabilizing two adjacent vertebrae 25 and 26. FIG. 9a shows the spine in an unbent or straight position, while FIG. 9b shows the spine in a bent forward position.

A first pedicle screw 27 is screwed into the posterior pedicle of the upper vertebra 25 and a second pedicle screw 28 is screwed into the posterior pedicle of the lower vertebra 26. The fixed coupling 13 extends through a hole in the head of the pedicle screw 28 and the floating coupling 15 extends through a hole in the head of the pedicle screw 27. Each coupling is retained in the hole of the corresponding pedicle screw head by frictional engagement, cement, or any other suitable means.

When the spine bends, as shown for example in FIG. 9b, the floating coupling 15 is pulled away from the fixed coupling 13, causing the spring 12 to extend. This allows natural pivoting motion of the disc, which includes some traction (expansion) and compression. FIG. 9c shows typical standard rod that does not allow for expansion of the pedicle screws and therefore compressing the disc all around in an un-natural manor.

The flexible rod assemblies 30, 40, 50, 60, 70, 80, and 90 are installed with their couplings adjacent corresponding vertebrae and engaged with corresponding pedicle screws, in the same manner as is described above with regard to assembly 10.

### Alternate Spring Construction

Instead of being a coiled wire, each of the springs used in the various embodiments may be in the form of a tube which is spirally (or otherwise) cut to allow it to act as a spring. A spring of this type exhibits extension properties as well as bending properties, allowing it to serve the same purpose as the core rod 11, for example. Such a spiral cut tube spring may alternatively serve the same purpose as the spring 12, for example. Another alternative would be to substitute a single spiral cut tube member for both the core rod and the surrounding spring, to perform both functions.

The cutting of the spring, which is preferably of a biologically inert metal, can be done with a laser, water jet, or milling machine. The pattern of the cuts may be a jigsaw type configuration similar to the configuration described in U.S. Pat. No. 6,053,922. The spring may also be of plastic or a polymer.

The core rods 1A through 1E are resilient and may comprise a relatively stiff wire wound flexible shaft, a solid rod, a tube, or a wire rope.

Except for the seventh embodiment shown in FIGS. 7a to 7c, each core rod is connected to a surrounding or inner spring and associated couplings in such a way that when the core rod is bent, the spring extends and the associated couplings move away from each other, with the core rod generating a force tending to return the core rod to its unbent position and the spring generating a force tending to return the couplings to their unextended position.

A flexible, resilient shaft core rod is composed of a central or mandrel wire, upon which are wound one or more successive helical wire layers, each layer being wound with a pitch direction opposite to that of the preceding layer. Flexible shafts of this type are shown, for example, in U.S. Pat. Nos. 571,869 to Stow; 1,905,197 to Webb; 1,952,301 to Webb; 2,142,497 to Clendenin; 2,401,100 to Pile; 2,875,597 to Neubauer; 3,274,846 to Forster; 4,112,708 to Fukuda; and 5,288,270 to Ishikawa.

Where the core rod is solid, it is made of a compliant flexible and resilient material.

Where the core rod is a wire rope, it consists of at least two stranded wires.

The flexible spinal fixation rod assemblies 20, 30, 40, 50, 60, 70, 80, and 90 should be of a diameter comparable to those of currently used spinal fixation rods, with the couplings preferably being dimensioned to fit with standard pedicle screws.

A small clearance is provided between the outer cylindrical surface of the core rod and the inner cylindrical surface of the surrounding spring, the amount of which clearance is not critical.

If desired, the spring may be dimensioned to cover one or more of the adjacent couplings.

Where the core rod is a tube, the spring may be disposed coaxially inside or outside of the tube.

The spring should have an axial spring rate so as to be easily extended without undue effort on the part of the patient, while applying sufficient axial force when extended so as to provide the desired amount of stabilization.

The couplings can be affixed to the spring and/or core using, but not limited to, one or more of the following methods: swaging, welding, brazing, pinning, and other mechanical fastening.

Each optional end stop may be attached to or formed as an integral part of the corresponding core adjacent one end thereof. This end stop may be affixed to the core end, that is, the end having a sliding coupling, by various means, such as but not limited to, welding, brazing, swaging, or pinning.

In a preferred embodiment of a core rod wherein it comprises a resilient flexible shaft, the shaft consists of a central mandrel wire of about 0.045 inch diameter around which are spiral wound six (6) layers of about 0.045 inch diameter wire, each layer being wound in a pitch direction opposite to that of the underlying layer. However, the shaft construction may consist of fewer or more layers and of wires of different diameters. The central or mandrel wire is made of a flexible material.

In addition to stainless steel or titanium alloys, polymer, or plastic, the core may comprise a wire rope or nickel-titanium shaped memory material, or any combination of flexible and bendably resilient materials or assemblies that would provide the required bending compliance and resilience.

Where the core is a wire rope, it may be made of stainless steel, titanium alloys, plastic, polymer or similar materials. Where the core is a tube, it may be made of a flexible and resilient material selected from the group consisting of, but not limited to nitnanol, carbon fiber composite, plastic or polymer.

The relatively axially stiff, yet relatively flexible and resilient (in bending) core is designed to have relatively high buckling strength, so as to support and stabilize the spinal column it is spanning. The bending strength is designed to be moderate, so as to allow bending and twisting of the spine through a radius of about 14 inches which represents a typical spinal bending radius, while maintaining spinal stability. The core provides most of the column strength for the flexible spinal fixation rod assembly.

The spring is designed with a relatively low bending strength and low axial extension spring rate, so as to permit free axial extension when the rod is flexed during bending. The preferred embodiment of the spring consists of closely helically wound wire of about 0.032 inch diameter. The spring material is preferably but not limited to stainless steel or titanium alloy grades used in medical implants. The spring wire size and strength is selected to provide a relatively low axial extension spring rate.

The spring may be made of round wire, flat wire, square wire, or any other shaped wire, in order to create column strength for axial rigidity during compression while also allowing axial stretch during decompression (extension) or bending of the spine. Such shaped wire may be in the form of a cross-sectional “V” shape wherein the wire coils nest into one another to provide greater column strength upon compression.

Multiple wires or multiple coaxial coils or springs can also be employed to achieve the desired results. Each coil or spring may be fabricated from a polymer that has column strength and axial compliance in tension.

A spring does not necessarily need to be used in every embodiment of the flexible spinal fixation rod. In the assembly 80, the spring is left out. The installation of the rod to the vertebrae using the couplings is done so as to keep the couplings in a compressed condition, i.e. with axial compression stress on the rod in its intended or straight installed configuration. Thus as the patient bends the spine, the couplings slide axially on the core 11J and return to the compressed initial or intended configuration when the patient straightens the spine.

The optional end stop limits the amount of axial extension, if such a limitation is required or desired.

In lieu of the couplings, the pedicle screws may be attached directly to the spring when the spring is designed with enough strength to allow this.

The length of traverse of the floating or sliding couplings should be greater than the maximum extension amount, so that the sliding end of the core is always disposed within the coupling.

Axial extension of the pedicle screws on the rod allows the spine to flex and pivot naturally on the spinal disc as shown in FIG. 9b with normal traction (extension) and compression, without compressing the entire spinal disc unnaturally, as shown in FIG. 9c as may occur with use of typical currently used flexible spinal rods that don't allow for extension of the pedicle screws.

For each of the embodiments, a flexible conformal coating or covering may be applied over the outer spring or coil, to minimize or eliminate tissue ingrowth to the assembly. This coating or covering can take the form of a flexible polymer applied as a coating (such as a paint or ‘rubber’ coating) or as a covering, such as a shrink tube covering. Covering materials can vary widely, as long as they are flexible and biocompatible for the intended use. They may be, but are not limited to, silicone, vinyl, urethane, polyvinyl chloride, or another polymer or elastomer typically used in implants. The covering may also comprise a relatively thin metal foil.

The spring, as well as the core, may alternatively be fabricated with an oval cross-section, to provide directional properties to the assembly. This may be of advantage for correction of certain spine instabilities. The oval cross-section may be incorporated by any suitable manufacturing technique for generating non-circular cross-sections, such as by swaging or pressing of a spring of circular cross-section.

1. A flexible spinal fixation rod assembly comprising:
an elongated resilient inner member;
an elongated resilient outer member coaxial with said inner member,
one of said members being relatively stiff in bending and axially stiff so as to provide substantial column strength, and the other of said members being relatively compliant in bending and adapted to provide substantial additional column strength;
a first end of the inner member being coupled to an adjacent first end of the outer member so that there is essentially no relative movement between said first ends;
first coupling means operatively associated with the first ends of said members and adapted to be connected to a first pedicle screw; and
second coupling means operatively associated with the second ends of said members and adapted to be connected to a second pedicle screw, said second coupling means allowing the inner and outer members to extend independently of each other.

2. The assembly according to claim 1, wherein said inner member is a rod or tube and said outer member is a spring.

3. The assembly according to claim 1, wherein said inner member is a spring and said outer member is a tube.

4. The assembly according to claim 2, wherein said first coupling means is affixed to said first ends of said members and said second coupling means is affixed to said rod or tube.

5. The assembly according to claim 3, wherein said first coupling means is affixed to said first ends of said members and said second coupling means is affixed to said tube.

6. The assembly according to claim 4 or 5, wherein said second coupling means includes a stop element for limiting the extension of said spring.

7. A flexible spinal fixation rod assembly comprising:
a resilient elongated structure exhibiting a predetermined level of bending stiffness and a predetermined limit of axial extension, and means for attaching the ends of said structure to corresponding pedicle screws for attachment to corresponding vertebrae so as to span two or more vertebrae and allow limited extension axially when the spine is bent while providing compressive resistance when the spine is in a predetermined intended configuration,
said structure comprising a rod and a spring fixedly coupled to each other at one end and coupled to each other at the other end in such a way that the spring may extend independently of the rod.

8. The assembly according to claim 7, wherein said rod comprises a wire wound flexible shaft or wire rope coaxially surrounded by the spring.

9. The structure according to claim 8, wherein said spring comprises a spiral member cut from a tube.

10. A flexible spinal fixation rod assembly comprising:
an inner, bendably resilient, relatively bendably stiff wire wound flexible shaft comprising a central or core wire upon which two or more wire layers are wound in opposite pitch directions;
a relatively bendably compliant spring surrounding a major portion of the flexible shaft; and
coupling means operationally associated with each end of the shaft and the spring in such a way that when the shaft is bent, at least a major portion of the spring extends independently of the shaft, with the shaft generating a force tending to return the core rod to the unbent configuration thereof and the spring generating a force tending to return the couplings to the unextended configuration thereof.

11. (canceled)

12. A flexible spinal fixation rod assembly comprising:
an inner, bendably resilient, relatively stiff wire wound flexible shaft comprising a central or core wire upon which two or more wire layers are wound in opposite pitch directions;
an elongated relatively bendably compliant spring surrounding a major portion of the flexible shaft;
a fixed coupling affixed to adjacent ends of said shaft and spring; and
a floating coupling affixed to the other end of said shaft, whereby when the shaft is bent, at least a major portion of the spring extends independently of the shaft with the shaft generating a force tending to return the shaft to the unbent configuration thereof and the spring generating a force tending to return the couplings to the unextended configuration thereof.

13. A flexible spinal fixation rod assembly comprising:
coaxial elongated spring and rod members having substantially different bending compliance, said rod member being bendably resilient; and
means for coupling the ends of the rod to posterior pedicles of corresponding vertebrae, so that the rod does not provide any significant resistance to extension and when the rod is bent, at least a major portion of the spring extends independently of the rod with the rod generating a force tending to return the rod to its unbent configuration thereof and the spring generating a force tending to return the couplings to there unextended configuration thereof.

14. The assembly according to claim 13, further comprising one or more additional coaxial spring and rod members operatively associated with said first mentioned spring and rod members, and means for coupling the ends of said additional members to posterior pedicles of corresponding vertebrae.

15. A flexible spinal fixation rod assembly comprising:
an elongated resilient inner member;
an elongated resilient outer member coaxial with said inner member,
one of said members being relatively stiff and the other of said members being relatively compliant;
a first end of the inner member being coupled to an adjacent first end of the outer member so that there is essentially no relative movement between said first ends;
first coupling means operatively associated with the first ends of said members and adapted to be connected to a first pedicle screw; and
second coupling means operatively associated with the second ends of said members and permitting relative movement of the second end of one member with respect to the second end of the other member so that said one member does not provide any significant resistance to extension of the assembly, and adapted to be connected to a second pedicle screw.

16. A flexible spinal fixation rod assembly comprising:
coaxial resilient elongated inner and outer members having different bending compliance; and
means for coupling the ends of one of said members to posterior pedicles of corresponding vertebrae, so that when said one member is bent, at least a major portion of the other member extends independently of said one member and urges said one member back toward the initial configuration.
